(19) United States
(12) Patent Application Publication
(10) Pub. No.: US 2018/0036020 A1
(43) Pub. Date: Feb. 8, 2018

(54) DEVICES AND METHODS FOR APPLYING ANTIGENS FOR ALLERGY TESTING

(71) Applicants: Ronald BARSHOP, Houston, TX (US); Jeff JACKSON, San Antonio, TX (US)

(72) Inventors: Ronald BARSHOP, Houston, TX (US); Jeff JACKSON, San Antonio, TX (US)

(21) Appl. No.: 15/666,537

(22) Filed: Aug. 1, 2017

Related U.S. Application Data
(60) Provisional application No. 62/369,874, filed on Aug. 2, 2016.

(51) Int. Cl. A61B 17/20; C40B 40/02

(52) U.S. Cl. CPC A61B 17/205; C40B 40/02; A61B 5/00

(57) ABSTRACT

Devices and methods for applying antigens for allergy testing. In certain embodiments the device comprises an elongated body comprising a first end and a second end and a curved lower surface extending between the first and second ends. The device may also comprise a substantially planar upper surface extending between the first and second ends, and a plurality of prongs extending from the curved lower surface. In specific embodiments, the plurality of prongs comprises at least a first prong and a second prong, and the angle between the first prong and the second prong is between 45 and 90 degrees when the device is viewed from the first end.

### CROSS-REFERENCE TO RELATED APPLICATION

This application claims priority to U.S. Provisional Patent Application Ser. No. 62/369,874 filed Aug. 2, 2016, the contents of which are incorporated by reference herein.

### BACKGROUND INFORMATION

Existing devices and methods for applying antigens to a skin surface for allergy testing include numerous shortcomings. For example, existing devices make it difficult for a user to apply a consistent and desired amount of pressure to the skin surface. In addition, existing devices and methods can present challenges in confirming the antigens are applied to the device in the proper location and are applied to the skin surface effectively.

As discussed in further detail below, exemplary embodiments of the present disclosure address shortcomings of existing devices and methods, and accordingly provide notable benefits in comparison to such systems and methods.

### SUMMARY

Exemplary embodiments of the present disclosure include devices and methods for applying antigens for allergy testing.

Exemplary embodiments include a device for applying antigens to a subject skin surface for allergy testing, the device comprising: an elongated body comprising a first end and a second end; a curved lower surface extending between the first and second ends; a substantially planar upper surface extending between the first and second ends; and a plurality of prongs extending from the curved lower surface. In certain embodiments, the plurality of prongs comprises at least a first prong and a second prong, and the angle between the first prong and the second prong is between 45 and 90 degrees when the device is viewed from the first end. In particular embodiments, each of the plurality of prongs comprises a first end distal to the curved lower surface, and each of the plurality of prongs comprises a plurality of tines at the first end. In some embodiments, the plurality of tines at the first end of each prong comprises eight tines. In specific embodiments, the plurality of tines are triangle-shaped.

In certain embodiments, the plurality of tines are distributed around an outer perimeter of the first end of each prong. In particular embodiments, the outer perimeter is circular. In some embodiments, the first end comprises a first textured gripping surface, and the second end comprises a second textured gripping surface. In specific embodiments, the curved lower surface comprises a third textured gripping surface extending between the first end and the second end, and the curved lower surface comprises a fourth textured gripping surface extending between the first end and the second end.

In certain embodiments, the curved lower surface is a semi-circular shape when viewed from the first end. Particular embodiments further comprise an antigen receptacle configured to couple to the substantially planar upper surface. In some embodiments, the antigen receptacle comprises a plurality of antigen containers. In specific embodiments, the antigen receptacle comprises a first plurality of numeric indicators, where: each numeric indicator in the first plurality of numeric indicators indicates a position of an antigen container; the elongated body comprises a second plurality of numeric indicators, wherein each numeric indicator in the second plurality of numeric indicators indicates a position of prong; and each numeric indicator in the first plurality of numeric indicators has an equivalent numeric indicator in the second plurality of numeric indicators.

In certain embodiments, each of the antigen containers is covered by a seal. In particular embodiments, the seal comprises a metallic foil. In some embodiments, the plurality of prongs are configured to align with the plurality of antigen containers when the antigen receptacle is positioned such that the seal is proximal to the curved lower surface. In specific embodiments, the antigen receptacle is configured to couple to the substantially planar upper surface via a first snap member proximal to the first end and via a second snap member proximal to the second end. In certain embodiments, the elongated body comprises a first orientation indicator directed toward the first end of the elongated body; and the antigen receptacle comprises a second orientation indicator directed toward the first end of the elongated body when the antigen receptacle is coupled to the substantially planar upper surface.

In particular embodiments, the elongated body comprises a first rocking member proximal to the first end, and a second rocking member proximal to the second end. In some embodiments, the plurality of prongs comprises ten prongs and in specific embodiments, the plurality of prongs comprises twelve prongs.

Certain embodiments include a method of applying antigens to a subject skin surface for allergy testing, where the method includes obtaining a device comprising: an elongated body comprising a first end and a second end; a curved lower surface extending between the first and second ends; a substantially planar upper surface extending between the first and second ends; and a plurality of prongs extending from the curved lower surface. In certain embodiments, the plurality of prongs comprises at least a first prong and a second prong; and the angle between the first prong and the second prong is between 45 and 90 degrees when the device is viewed from the first end. Certain embodiments include: applying a plurality of antigens to the plurality of prongs of the device, wherein a single antigen is applied to each prong of the plurality of prongs; gripping the elongated body of the device; engaging the curved lower surface of the device to the subject skin surface; and rotating the device along an axis extending between the first end and the second end of the elongated body such that the first prong initially contacts the subject skin surface and the second prong subsequently contacts the subject skin surface after the first prong.

In particular embodiments, applying a plurality of antigens to the plurality of prongs of the device comprises: obtaining an antigen receptacle comprising a plurality of antigen containers, wherein each of the antigen containers contains an antigen and is covered by a seal; positioning the antigen receptacle such that antigen containers are proximal to the curved lower surface of the device; aligning the plurality of prongs with the plurality of antigen containers; and piercing the seal with the plurality of prongs so that each prong is inserted into an antigen container.

In certain embodiments, the antigen receptacle is initially coupled to the device, and further comprise separating the antigen receptacle from the device prior to positioning the antigen receptacle such that antigen containers are proximal to the curved lower surface of the device. In particular embodiments, the antigen receptacle is initially coupled to the substantially planar upper surface. In some embodiments, the device comprises a first orientation indicator, and the antigen receptacle comprises a second orientation indicator. Specific embodiments, further comprise positioning the antigen receptacle such that the first orientation indicator and the second orientation indicator are both oriented in a same direction.

In certain embodiments, the antigen receptacle comprises a first plurality of numeric indicators, where each numeric indicator in the first plurality of numeric indicators indicates a position of an antigen container; and the elongated body comprises a second plurality of numeric indicators, wherein each numeric indicator in the second plurality of numeric indicators indicates a position of prong; and each numeric indicator in the first plurality of numeric indicators has an equivalent numeric indicator in the second plurality of numeric indicators.

Particular embodiments, further comprise verifying each numeric indicator in the first plurality of numeric indicators is proximal to the equivalent numeric indicator in the second plurality of numeric indicators. In some embodiments, the first end of the device comprises a first textured gripping surface; the second end of the device comprises a second textured gripping surface; the curved lower surface of the device comprises a third textured gripping surface extending between the first end and the second end; and the curved lower surface of the device comprises a fourth textured gripping surface extending between the first end and the second end.

In some embodiments, gripping the elongated body of the device comprises gripping one or more of the first textured gripping surface, the second textured gripping surface, the third textured gripping surface and the fourth textured gripping surface. In particular embodiments, the elongated body comprises a first rocking member proximal to the first end and comprises a second rocking member proximal to the second end; and the method further comprises verifying a desired amount of pressure is being applied to the subject skin surface by confirming that the first and second rocking members make an impression on the subject skin surface while rotating the device along the axis extending between the first end and the second end of the device. In some embodiments, the plurality of prongs comprises ten prongs and in specific embodiments, the plurality of prongs comprises twelve prongs.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 illustrates an exploded side view of a first embodiment of a device for applying antigens for allergy testing.

FIG. 2 illustrates an end view of a first component of the embodiment of FIG. 1.

FIG. 3 illustrates a top view of the component of FIG. 2.

FIG. 4 illustrates a top view of a second component of the embodiment of FIG. 1.

FIG. 5 illustrates a partial perspective view of a feature of the embodiment of FIG. 1.

FIG. 6 illustrates an end view of the feature of FIG. 5.

FIG. 7 illustrates an exploded side view of a second embodiment of a device for applying antigens for allergy testing.

FIG. 8 illustrates a top view of a second component of the embodiment of FIG. 7.

### DETAILED DESCRIPTION OF ILLUSTRATIVE EMBODIMENTS

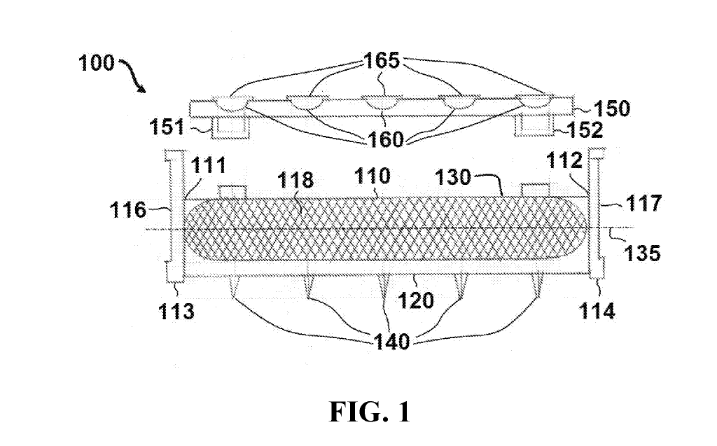

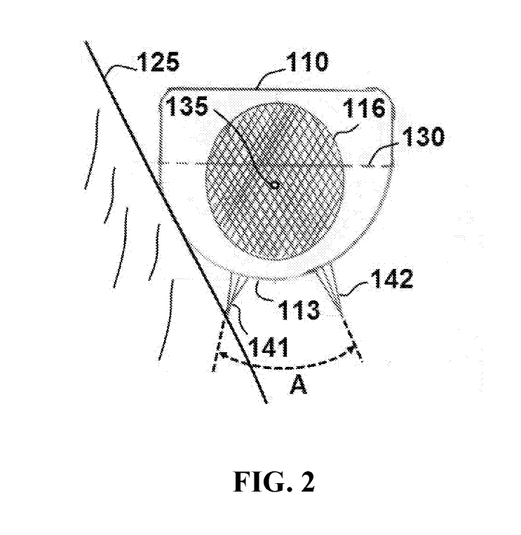

Referring initially to FIG. 1, a device 100 for applying antigens to a subject skin surface for allergy testing is shown with an elongated body 110 comprising a first end 111, a second end 112, a curved lower surface 120, and a substantially planar upper surface 130, each extending between the first end 111 and second end 112. As shown in FIG. 1, device 100 also comprises a plurality of prongs 140 extending from curved lower surface 120. As shown in the end view of FIG. 2, prongs 140 are arranged in two rows such that an angle A between a prong in the first row and a prong in the second row is between 45 and 90 degrees when device 100 is viewed from first end 111. In specific embodiments, the angle between the rows of prongs may be approximately 45, 50, 55, 60, 65, 70, 75, 80, or 85 degrees. As explained in more detail below, this configuration can enable a user to effectively apply antigens to a subject skin surface for allergy testing.

In the embodiment shown, device 100 also comprises an antigen receptacle 150 configured to couple to substantially planar upper surface 130 via a first snap member 151 proximal to first end 111 and a second snap member 152 proximal to second end 112. Antigen receptacle 150 can be initially coupled to elongated body 110 (e.g., for shipment purposes) and then separated prior to use.

Antigen receptacle 150 comprises a plurality of antigen containers 160 covered by a seal 165. In particular embodiments, seal 165 may comprise individual metallic foil covers for each antigen container 160. In other embodiments, seal 165 may comprise a single cover layer that covers all of antigen containers 160. As explained in further detail below, an antigen from each antigen container 160 can be applied to each prong 140 prior to applying the antigens to a subject skin surface. For example, antigen receptacle 150 can be positioned such that seal 165 is proximal to curved lower surface 120 and prongs 140 are aligned with antigen containers 160. Elongated body 110 and antigen receptacle 150 can then be engaged such that prongs 140 pierce seal 165 and each prong 140 is inserted into an antigen container 160.

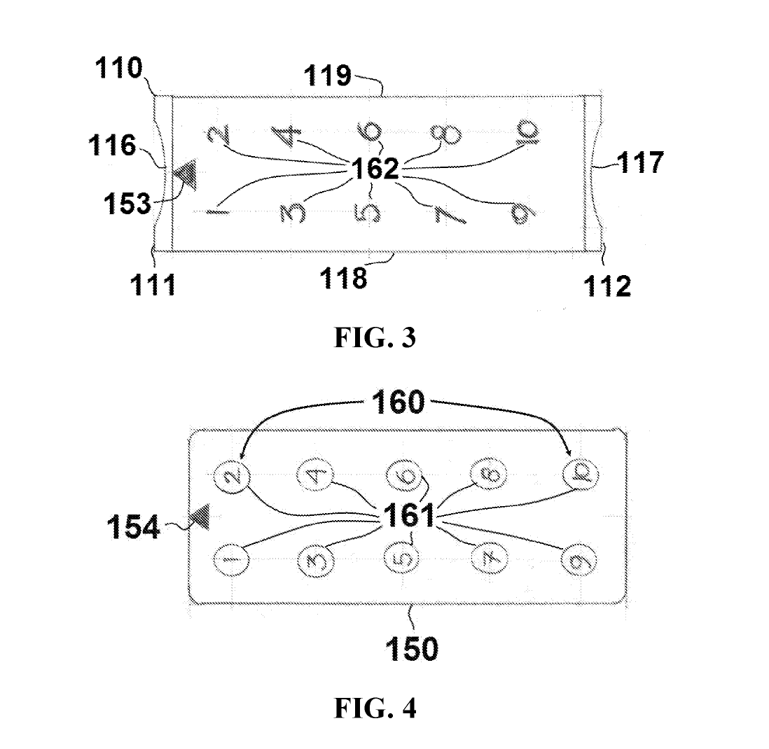

Device 100 may also comprise features to assist a user in aligning elongated body 110 and antigen receptacle 150 to ensure prongs 140 and antigen containers 160 are properly aligned. Referring now to FIGS. 3 and 4, for example, antigen receptacle 150 may comprise a first plurality of numeric indicators 161, each indicating a position of an antigen container 160. In addition, elongated body 110 may comprise a second plurality of numeric indicators 162, each indicating a position of prong in the plurality of prongs 140. In the embodiment shown, each numeric indicator in the first plurality of numeric indicators 161 has an equivalent numeric indicator in the second plurality of numeric indicators 162. This can assist a user in confirming elongated body 110 and antigen receptacle 150 are properly positioned such that the corresponding antigen containers 160 and prongs 140 are aligned when antigens are applied to prongs 140.

In addition, elongated body 110 may comprise a first orientation indicator 153 (e.g. an arrow) directed toward first end 111. Antigen receptacle 150 may also comprise a second orientation indicator 154 directed toward first end 111 when the antigen receptacle 150 is coupled to substantially planar upper surface 130. Orientation indicators 153 and 154 can be used to verify that elongated body 110 and antigen receptacle 150 are properly aligned when prongs 140 are used to pierce seal 165 and apply antigens to prongs 140.

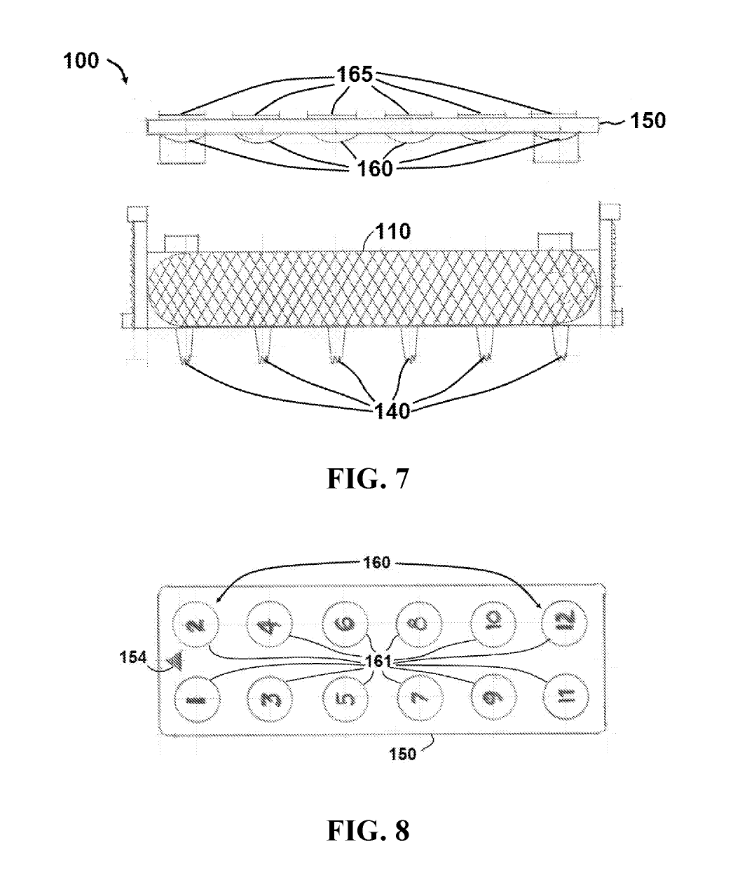

While the embodiment shown in FIGS. 1-4 illustrates an antigen receptacle with ten prongs and antigen containers, other embodiments may comprise a different number of prongs and antigen containers. For example, as shown in FIGS. 7-8, device 100 may comprise an elongated body 110 with a plurality of prongs 140 that comprises twelve prongs. In addition, antigen receptacle 150 comprises twelve antigen containers 160 and seals 165. Similar to the embodiment of FIG. 4, antigen receptacle 150 also comprises numeric indicators 161 and orientation indicator 154. In other aspects, the embodiment shown in FIGS. 7-8 is equivalent to the embodiment of FIGS. 1-4. For sake of clarity and to avoid redundancy, the remaining features and components in the embodiment of FIGS. 7-8 are not labeled with reference numbers. Embodiments with a higher number of prongs and antigen containers can be less costly, allow less storage and the test is faster in and out for the patient. For example, such embodiments can reduce the testing time and costs by allowing a greater number of antigens to be applied at one time. Still other embodiments may comprise different numbers of antigen containers and prongs.

After antigens have been applied to prongs 140, elongated body 110 can be used to apply antigens to a subject skin surface for allergy testing. Elongated body comprises also comprises features to assist a user in applying the antigens to the subject skin surface. For example, first end 111 may comprise a first textured gripping surface 116 and second end 112 may comprise a second textured gripping surface 117 to assist a user in grasping elongated body 110 when applying antigens to the subject skin surface. In certain embodiments, curved lower surface 120 comprise a third textured gripping surface 118 and a fourth textured gripping surface 119 extending between first end 111 and second end 112. While the fourth textured gripping surface is not visible in the figures, it is understood to be equivalent to third textured gripping surface 118 but on the opposing side of curved lower surface 120 from third textured gripping surface 118.

During application of antigens to a subject skin surface, a user can grip any of the textured gripping surfaces and engage elongated body 110 with the skin surface. For example, referring now to FIG. 2, elongated body 110 can be positioned so that curved lower surface 120 initially engages skin surface 125 on one side of prongs 140. Elongated body 110 can then be rotated along an axis 135 extending between first end 111 and second end 112 of elongated body 110 such that a first prong 141 initially contacts skin surface 125 and a second prong 142 subsequently contacts 125 skin surface after first prong 141. In exemplary embodiments, curved lower surface can extend through an arc of approximately 180 degrees to allow a user to roll elongated body 110 in an even manner and apply uniform pressure to skin surface 125 in a natural and ergonomic motion. In particular embodiments, elongated body 110 comprises rocking members 113 and 114 proximal to first end 111 and second end 112, respectively. In the embodiment shown, rocking members 113 and 114 extend slightly below curved lower surface 120. During use, rocking members 113 and 114 can be used to confirm that a proper amount of pressure is being applied to skin surface 125 by elongated body 100 during the application of antigens. For example, a user can verify the desired amount of pressure is being applied by confirming that rocking members 113 and 114 are making an impression skin surface 125.

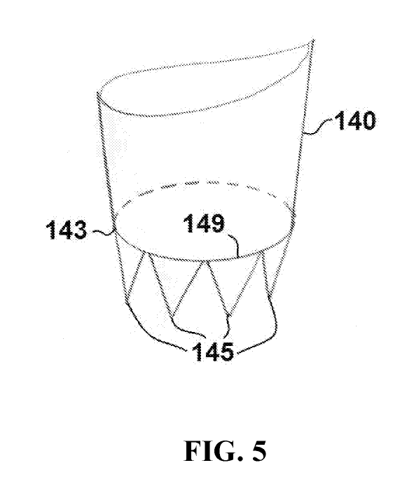

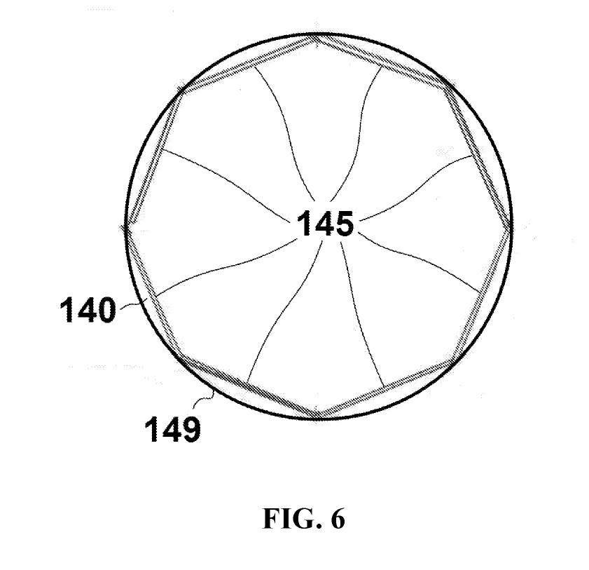

Referring now to FIGS. 5 and 6, more detailed views of a prong 140 are shown. In FIG. 5, a partial perspective view is shown of prong 140 having a first end 143 comprising a plurality of tines 145. In this embodiment, tines 145 are triangle-shaped and are distributed around an outer perimeter 149 of first end 143 of prong 140. FIG. 6 illustrates an end view of prong 140, which shows eight tines 145 in this embodiment. During application of antigens to skin surface 125, tines 145 will make an impression in skin surface 125 which can provide visual confirmation to a user that the desired amount of pressure is being applied to elongated body 110.

As discussed herein, exemplary embodiments of the present disclosure address shortcomings of existing devices and methods.

Accordingly, embodiments of this disclosure provide notable benefits in comparison to such devices and methods.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:

U.S. Pat. No. 2,522,309

U.S. Pat. No. 4,205,689

U.S. Pat. No. 4,607,632

U.S. Pat. No. 4,711,247

U.S. Pat. No. 5,647,371

U.S. Pat. No. 5,746,700

U.S. Pat. No. 5,931,794

U.S. Pat. No. 6,095,988

U.S. Pat. No. 6,554,777

U.S. Pat. No. 7,631,765

U.S. Patent 20100030100

U.S. Patent Publication 20150289794

U.S. Patent Publication 20150126898

U.S. Patent Publication 2015034251

Webpage available at: http://www.allergimed.com/allergitesting/about-allergitest/

1. A device for applying antigens to a subject skin surface for allergy testing, the device comprising:
an elongated body comprising a first end and a second end;
a curved lower surface extending between the first and second ends;
a substantially planar upper surface extending between the first and second ends; and
a plurality of prongs extending from the curved lower surface, wherein:
the plurality of prongs comprises at least a first prong and a second prong; and
the angle between the first prong and the second prong is between 45 and 90 degrees when the device is viewed from the first end.

2. The device of claim 1 wherein:
each of the plurality of prongs comprises a first end distal to the curved lower surface; and
each of the plurality of prongs comprises a plurality of tines at the first end.

3. The device of claim 2 wherein the plurality of tines at the first end of each prong comprises eight tines.

4. The device of claim 2 wherein the plurality of tines are triangle-shaped.

5. The device of claim 2 wherein the plurality of tines are distributed around an outer perimeter of the first end of each prong.

6. The device of claim 5 wherein the outer perimeter is circular.

7. The device of claim 1 wherein:
the first end comprises a first textured gripping surface; and
the second end comprises a second textured gripping surface.

8. The device of claim 7 wherein:
the curved lower surface comprises a third textured gripping surface extending between the first end and the second end; and
the curved lower surface comprises a fourth textured gripping surface extending between the first end and the second end.

9. The device of claim 1 wherein the curved lower surface is a semi-circular shape when viewed from the first end.

10. The device of claim 1 further comprising an antigen receptacle configured to couple to the substantially planar upper surface.

11. The device of claim 10 wherein the antigen receptacle comprises a plurality of antigen containers.

12. The device of claim 10, wherein:
the antigen receptacle comprises a first plurality of numeric indicators, wherein each numeric indicator in the first plurality of numeric indicators indicates a position of an antigen container;
the elongated body comprises a second plurality of numeric indicators, wherein each numeric indicator in the second plurality of numeric indicators indicates a position of prong; and
each numeric indicator in the first plurality of numeric indicators has an equivalent numeric indicator in the second plurality of numeric indicators.

13. The device of claim 11 wherein each of the antigen containers is covered by a seal.

14. The device of claim 13 wherein the seal comprises a metallic foil.

15. The device of claim 13 wherein:
the plurality of prongs are configured to align with the plurality of antigen containers when the antigen receptacle is positioned such that the seal is proximal to the curved lower surface.

16. The device of claim 10 wherein the antigen receptacle is configured to couple to the substantially planar upper surface via a first snap member proximal to the first end and via a second snap member proximal to the second end.

17. The device of claim 10 wherein:
the elongated body comprises a first orientation indicator directed toward the first end of the elongated body; and
the antigen receptacle comprises a second orientation indicator directed toward the first end of the elongated body when the antigen receptacle is coupled to the substantially planar upper surface.

18. The device of claim 1 wherein the elongated body comprises:
a first rocking member proximal to the first end; and
a second rocking member proximal to the second end.

19. The device of claim 1 wherein the plurality of prongs comprises ten prongs.

20. The device of claim 1 wherein the plurality of prongs comprises twelve prongs.

21. A method of applying antigens to a subject skin surface for allergy testing, the method comprising:
obtaining a device comprising:
an elongated body comprising a first end and a second end;
a curved lower surface extending between the first and second ends;
a substantially planar upper surface extending between the first and second ends; and
a plurality of prongs extending from the curved lower surface, wherein:
the plurality of prongs comprises at least a first prong and a second prong; and
the angle between the first prong and the second prong is between 45 and 90 degrees when the device is viewed from the first end;
applying a plurality of antigens to the plurality of prongs of the device, wherein a single antigen is applied to each prong of the plurality of prongs;
gripping the elongated body of the device;
engaging the curved lower surface of the device to the subject skin surface; and
rotating the device along an axis extending between the first end and the second end of the elongated body such that the first prong initially contacts the subject skin surface and the second prong subsequently contacts the subject skin surface after the first prong.

22. The method of claim 21 wherein applying a plurality of antigens to the plurality of prongs of the device comprises:
obtaining an antigen receptacle comprising a plurality of antigen containers, wherein each of the antigen containers contains an antigen and is covered by a seal;
positioning the antigen receptacle such that antigen containers are proximal to the curved lower surface of the device;
aligning the plurality of prongs with the plurality of antigen containers; and
piercing the seal with the plurality of prongs so that each prong is inserted into an antigen container.

23. The method of claim 22 wherein the antigen receptacle is initially coupled to the device, and further comprising separating the antigen receptacle from the device prior to positioning the antigen receptacle such that antigen containers are proximal to the curved lower surface of the device.

24. The method of claim 23 wherein the antigen receptacle is initially coupled to the substantially planar upper surface.

25. The method of claim 22 wherein:
the device comprises a first orientation indicator; and
the antigen receptacle comprises a second orientation indicator.

26. The method of claim 25 further comprising:
positioning the antigen receptacle such that the first orientation indicator and the second orientation indicator are both oriented in a same direction.

27. The method of claim 26, wherein:
the antigen receptacle comprises a first plurality of numeric indicators, wherein each numeric indicator in the first plurality of numeric indicators indicates a position of an antigen container; and
the elongated body comprises a second plurality of numeric indicators, wherein each numeric indicator in the second plurality of numeric indicators indicates a position of prong; and
each numeric indicator in the first plurality of numeric indicators has an equivalent numeric indicator in the second plurality of numeric indicators.

28. The method of claim 27 further comprising:
verifying each numeric indicator in the first plurality of numeric indicators is proximal to the equivalent numeric indicator in the second plurality of numeric indicators.

29. The method of claim 21 wherein:
the first end of the device comprises a first textured gripping surface;
the second end of the device comprises a second textured gripping surface;
the curved lower surface of the device comprises a third textured gripping surface extending between the first end and the second end; and
the curved lower surface of the device comprises a fourth textured gripping surface extending between the first end and the second end.

30. The method of claim 29 wherein gripping the elongated body of the device comprises gripping one or more of the first textured gripping surface, the second textured gripping surface, the third textured gripping surface and the fourth textured gripping surface.

31. The method of claim 21 wherein:
the elongated body comprises a first rocking member proximal to the first end and comprises a second rocking member proximal to the second end; and
the method further comprises verifying a desired amount of pressure is being applied to the subject skin surface by confirming that the first and second rocking members make an impression on the subject skin surface while rotating the device along the axis extending between the first end and the second end of the device.

32. The method of claim 21 wherein the plurality of prongs comprises ten prongs.

33. The method of claim 21 wherein the plurality of prongs comprises twelve prongs.
